(19) United States
(12) Patent Application Publication
(10) Pub. No.: US 2003/0139373 A1
(43) Pub. Date: Jul. 24, 2003

(54) Method for cancer therapy

(76) Inventors: Lars Holger Breimer, Chatham, NJ (US); Kapil Dhingra, Tenafly, NJ (US); Urvashi Hooda Dhingra, Cedar Grove, NJ (US); Steve Ritland, Paterson, NJ (US)

Correspondence Address: HOFFMANN-LA ROCHE INC., PATENT LAW DEPARTMENT, 340 KINGSLAND STREET, NUTLEY, NJ, 07110

(21) Appl. No.: 10/290,652

(22) Filed: Nov. 8, 2002

Related U.S. Application Data
(60) Provisional application No. 60/333,977, filed on Nov. 20, 2001.

(51) Int. Cl. A61K 31/7072; A61K 31/404

(52) U.S. Cl. 514/49; 514/414

(57) ABSTRACT

Disclosed are compositions and a method of treating a patient suffering from cancer. The method comprises administering to a patient over a period of up to about 15 days a first component and a second component. The first component consists of a pharmaceutical composition containing as an active ingredient a compound of formula I or a pharmaceutically acceptable salt or ester of said compound. The second component consists of an injection solution containing as an active ingredient gemcitabine. The amount of each component in the combination is such that the combination is therapeutically effective. The components are administered concomitantly or sequentially in a three week to four week treatment cycle for as long as the tumor remains under control and the patient tolerates the regimen. The compositions comprise an antineoplastic combination of a compound for formula I and gemcitabine.

This application claims priority of Provisional application Serial No. 60/333,977, filed Nov. 20, 2001.

### FIELD OF THE INVENTION

The present invention is directed to a method of cancer therapy by administering (i) a pharmaceutical composition containing a compound of formula

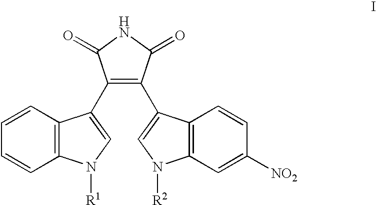

and (ii) a pharmaceutical composition containing gemcitabine (commercially available as Gemzar®, Lilly, Indianapolis, Ind.). The invention is also directed to a kit containing both of the above compositions, as well as to pharmaceutical compositions comprising an antineoplastic combination of a compound of formula I and gemcitabine.

### BACKGROUND OF THE INVENTION

The compounds of formula I below are known to be cell cycle inhibitors and apoptosis-inducers having antiproliferative and antitumor activity against a wide range of tumors, in particular in solid tumors such as breast and colon cancers. See, e.g. U.S. Pat. Nos. 5,057,614 and 6,048,887.

Gemcitabine is a nucleoside analog that exhibits antitumor activity by inhibiting DNA synthesis in S-phase of the cell cycle. See, e.g., Physicians' Desk Reference (54th Edition, 2000), pp1586 et seq.

It has now been discovered that compounds of formula I are effective in cancer therapy when administered in combination with gemcitabine without markedly increased toxicity. Moreover, because these two compounds exert antitumor effects by affecting different cellular mechanisms, a therapeutic combination of both compounds yields improved antitumor activity in certain tumors and/or prevents or delays resistance to drug therapy.

### SUMMARY OF THE INVENTION

The present invention relates to a method of treating a patient suffering with cancer comprising administering to the patient, either concomitantly or sequentially, a first component consisting of a pharmaceutical composition containing as an active ingredient a compound of formula I or a pharmaceutically acceptable salt or ester of said compound and a second component consisting of a pharmaceutical composition containing gemcitabine, the amount of each component being such that the combination of components is therapeutically effective.

The invention also relates to antitumor compositions comprising a compound of formula I and gemcitabine, the combination having a therapeutic activity which is greater than the individual components of said compositions without an increase in toxicity.

This combination of chemotherapeutic compounds is particularly useful in the treatment of lung, pancreatic, bladder, breast, colon, rectal, uterine and prostate cancers.

The invention permits reduction of the amount of at least one component (in comparison the amount typically given in monotherapy) while retaining a desirable therapeutic index. In preferred embodiments, the amount of both components (in comparison the amount typically given in monotherapy) is reduced affording reduced toxicity while still retaining a desirable therapeutic index.

In another aspect, the invention relates to a kit. The kit comprises a first component and a second component. The first component contains one or more oral unit dosage forms of an active ingredient selected from a compound of formula I or a pharmaceutically acceptable salt or ester of said compound. The second component contains one or more injectable unit dosage forms of gemcitabine.

In yet another aspect, the invention relates to pharmaceutical compositions comprising an antineoplastically effective amount of a combination of a compound of formula I and gemcitabine.

### DETAILED DESCRIPTION OF THE INVENTION

The terms “antineoplastic” or “antitumor” mean inhibiting or preventing the development, maturation or proliferation of malignant cells.

As used herein the term “concomitant” means administration of both components during the same 24 hour period, preferably within one or two hours of each other.

The term “pharmaceutically acceptable ester” of a compound of formula I means a conventionally esterified compound of formula I having a carboxyl group, which esters retain the biological effectiveness and properties of the compound of formula I.

The term “pharmaceutically acceptable salt” of a compound of formula I as used herein is any conventional salt or base addition salt that retains the biological effectiveness and properties of the compound of formula I and which is formed from a suitable non-toxic organic or inorganic acid or organic or inorganic base. Preferred salts are cationic salts, for example, of alkali metals, especially sodium salts.

As used herein “sequential” (as in sequential administration) means that one component is administered more than twenty four hours after the other component, preferably within 2-15 days of the other component.

As used herein, “therapeutically effective” or “effective amount” means an amount of drug, or combination or composition, which is effective for producing a desired therapeutic effect upon administration to a patient, for example, to stem the growth, or result in the shrinkage, of a cancerous tumor.

“Therapeutic index” is a well-recognized term of art and is an important parameter in the selection of anticancer agents for clinical trial. Therapeutic Index takes into consideration the efficacy, pharmacokinetics, metabolism and bioavailability of anticancer agents. See, e.g., J. Natl. Cancer Inst. 81(13): 988-94 (Jul. 5, 1989).

“Tumor control” means that the perpendicular diameters of measurable lesions has not increased by 25% or more from the last measurement. See, e.g., World Health Organization (“WHO”) Handbook for Reporting Results of Cancer Treatment, Geneva (1979).

The present invention is directed to a method of cancer therapy comprising the concomitant or, alternatively, sequential administration of two antineoplastic components. The first component of the present invention consists of a pharmaceutical composition containing as an active ingredient a compound of formula I, or a pharmaceutically acceptable salt or ester of said compound. The second component consists of a pharmaceutical composition containing as an active ingredient gemcitabine. The amount of each component in the combination is such that the combination is therapeutically effective to treat or ameliorate a cancerous tumor. The amount of each component administered according to the present method may, but does not have to be therapeutically effective by itself. That is, this invention specifically contemplates combinations wherein the amount of compound I and/or the amount of gemcitabine in the combination is less than a therapeutically effective amount as judged by the amounts recommended in monotherapy (i.e. a “suboptimal” amount).

The present invention is also directed to antineoplastic pharmaceutical compositions comprising an effective amount of a combination of a compound of formula I and gemcitabine, said compositions having therapeutic benefit as compared to monotherapy.

As used herein, a combination of antineoplastic agents exhibits a “therapeutic benefit” if it is therapeutically superior, that is less toxic and/or more efficacious against certain tumors than either of the constituents used alone (in monotherapy) and/or prevents or delays drug resistance in certain tumors.

The two components of the invention, that is a pharmaceutical composition containing a compound of formula I and a pharmaceutical composition containing gemcitabine, may be administered concomitantly or sequentially over such period of time so as to obtain maximum therapeutic effect. As is demonstrated below, when the components are administered sequentially, either component may be administered first. In a preferred embodiment, both components are administered concomitantly.

In accordance with the present invention, administration of the two components, concomitantly or sequentially, enhances the treatment of cancer as compared to administering each component independently in monotherapy. The combination effect results in an improved therapeutic index as compared to either agent alone while toxicity remains acceptable.

Preferably, the compound of formula I is administered to the patient in an oral unit dosage form, more preferably in capsule or tablet form. The second component, gemcitabine, is administered by parenteral, preferably by intravenous administration, in association with a compound of formula I as described herein.

The first component and the second component of the present invention are administered in any amount and for any duration that is effective to maintain or decrease tumor size.

In a preferred embodiment, administration of the composition containing a compound of formula I and the composition containing gemcitabine occur on the first day of a 21-28 day cycle (that is, a 3 to 4 weeks repeating cycle). The composition containing a compound of formula I is administered daily for up to about 14 days, preferably for about 7 days, and more preferably for about 4 days. The composition containing gemcitabine is administered preferably on days 1, 8 and 15 of a 21-28 day cycle, more preferably days 1 and 8 of a 21-28 day cycle, repeated for a total of up to about 16-24 doses.

In a preferred embodiment, the composition containing gemcitabine is administered on the first day of a 21-day cycle, preferably within about 16 hours after the administration of the composition containing a compound of formula I.

In another preferred embodiment, the composition containing a compound of formula I is administered on the first day of a 21-day cycle, preferably within about 8 hours after administration of the composition containing gemcitabine.

The course of a preferred cycle is 21 or 28 days, though cycles anywhere between about 21 to about 28 days are also effective and contemplated. When the composition containing a compound of formula I is administered for about 4 to about 7 days, a 21 day treatment cycle is preferred. When the composition containing a compound of formula I is administered for about 14 days, a 28 day treatment cycle is preferred. At the end of the 21-28 days of each cycle, the cycle of dosing is repeated for as long as clinically tolerated and the tumor is under control or until tumor regression. Tumor “control” is a well recognized clinical parameter, as defined above. In a preferred embodiment, the cycle of dosing is repeated for up to about 16 cycles.

In an alternative preferred embodiment, the composition containing gemcitabine is administered on days 1 and 8 of a 3 week (21 days) or 4 week (28 days) cycle, preferably a 3 week cycle.

The dose intensity of compound of formula I is from about 260 mg/m² week to about 750 mg/m²/week. The total overall dosage for the compound of formula I for a period of up to about 21-28 days is from about 780 mg/m² to about 3000 mg/m². A patient's body measurement in square meters (“m²”), this is a “BSA (body surface area”) measurement”, typically ranges from about 1.4 m² to about 2.2 m². Thus, the total amount of compound of formula I to be delivered in a treatment cycle (mg) is calculated as follows:

[Dose intensity(mg/m²/week)]×[BSA(m²)]×[number of weeks in treatment cycle]

The foregoing amount of compound of formula I is divided, preferably into equal doses (though this is not required), and administered daily, as a single dose or divided into two or more doses daily, preferably twice per day, most preferably at 12 hour intervals (“Q12” or “BID”). The length of preferred treatment cycle is from about 3 to about 4 weeks.

Preferably, the compound of formula I is administered twice daily over a period of about 1-14 days. Preferred therapeutic regimens for administration of compounds of formula I are summarized in Tables 1A-1C below.

TABLE 1A
PREFERED DOSAGE REGIMENS OF COMPOUNDS OF FORMULA 1:
THREE WEEK CYCLE

| | | Range Total | | | |
|---|---|---|---|---|---|
| | Dose Intensity | Dose/Cycle | | No. of days of | Individual Dose |
| | (mg/m²/week) | (mg/m²) | BSA Range (m²) | Dosing | (mg/m² BID) |
| Desired | 260-750 | 780-2250 | 1.4-2.2 | 4 | 95-285 |
| Preferred | 370-590 | 1110-1770 | 1.4-2.2 | 4 | 135-225 |

TABLE 1B
PREFERED DOSAGE REGIMENS OF COMPOUNDS OF FORMULA 1:
FOUR WEEK CYCLE

| | | Range Total | | | |
|---|---|---|---|---|---|
| | Dose Intensity | Dose/Cycle | | No. of days of | Individual Dose |
| | (mg/m²/week) | (mg/m²) | BSA Range (m²) | Dosing | (mg/m² BID) |
| Desired | 260-750 | 1040-3000 | 1.4-2.2 | 7 | 70-215 |
| Preferred | 370-590 | 1480-2360 | 1.4-2.2 | 7 | 100-170 |

TABLE 1C
PREFERED DOSAGE REGIMENS OF COMPOUNDS OF FORMULA 1:
FOUR WEEK CYCLE

| | | Range Total | | | |
|---|---|---|---|---|---|
| | Dose Intensity | Dose/Cycle | | No. of days of | Individual Dose |
| | (mg/m²/week) | (mg/m²) | BSA Range (m²) | Dosing | (mg/m² BID) |
| Desired | 260-750 | 1040-3000 | 1.4-2.2 | 14 | 35-110 |
| Preferred | 370-590 | 1480-2360 | 1.4-2.2 | 14 | 50-85 |

The dose intensity of gemcitabine is from about 300 mg/m²/week to about 900 mg/m²/week. The overall dosage of the gemcitabine is from about 1200 mg/m² to about 3600 mg/m², administered over a 21-28 day period. In a preferred embodiment, the gemcitabine is given as an i.v. infusion on days 1 and 8 of a 21 day cycle, and the regimen is then repeated for up to about 8 cycles.

In a second preferred embodiment, the gemcitabine is given as an i.v. infusion on days 1 and 8 of a 28 day cycle, and the regimen is then repeated for up to about 8 cycles.

In a third preferred embodiment, the gemcitabine is given as an i.v. infusion on days 1, 8, and 15 of a 28 day cycle, and the regimen is then repeated for up to about 8 cycles

While the doses of gemcitabine do not have to be equal, they typically are. In a most preferred embodiment, the total dose of gemcitabine is administered to the patient on days 1 and 8 of a 21 day cycle by approximately a short i.v. infusion, typically over a period of about 30 minutes.

Preferred therapeutic regimens for administration of gemcitabine are summarized in Tables 2A-2C below.

TABLE 2A
PREFERED DOSAGE REGIMENS OF GEMCITABINE: 3 WEEK CYCLE

| | | Range Total | | | |
|---|---|---|---|---|---|
| | Dose Intensity | Dose/Cycle | | No. of days of | Individual Dose |
| | (mg/m²/week) | (mg/m²) | BSA Range (m²) | Dosing | (mg/m² BID) |
| Desired | 460-800 | 1400-2400 | 1.4-2.2 | 2 (days 1, 8) | 700-1200 |
| Preferred | 530-670 | 1600-2000 | 1.4-2.2 | 2 (days 1, 8) | 800-1000 |

TABLE 2B
PREFERED DOSAGE REGIMENS OF GEMCITABINE: 4 WEEK CYCLE

| | | Range Total | | | |
|---|---|---|---|---|---|
| | Dose Intensity | Dose/Cycle | | No. of days of | Individual Dose |
| | (mg/m²/week) | (mg/m²) | BSA Range (m²) | Dosing | (mg/m² BID) |
| Desired | 300-600 | 1200-2400 | 1.4-2.2 | 2 (days 1, 8) | 600-1200 |
| Preferred | 400-500 | 1600-2000 | 1.4-2.2 | 2 (days 1, 8) | 800-1000 |

TABLE 2C
PREFERED DOSAGE REGIMENS OF GEMCITABINE: 4 WEEK CYCLE

| | | Range Total | | | |
|---|---|---|---|---|---|
| | Dose Intensity | Dose/Cycle | | No. of days of | Individual Dose |
| | (mg/m²/week) | (mg/m²) | BSA Range (m²) | Dosing | (mg/m² BID) |
| Desired | 450-900 | 1800-3600 | 1.4-2.2 | 3 (days 1, 8, 15) | 600-1200 |
| Preferred | 600-750 | 2400-3000 | 1.4-2.2 | 3 (days 1, 8, 15) | 800-1000 |

The dosage levels of each of the components may be modified by the physician to be lower or higher than that stated herein depending on the needs of the patient, and the reaction of the patient to the treatment. The dosages may be administered according to any dosage schedule determined by the physician in accordance with the requirements of the patient. For example, the dosages of each of the two components may be administered in single or in divided doses over a period of several days, or alternating daily schedules.

Preferably, four day treatment schedules are repeated every twenty one days, or as soon as permitted by recovery from toxicity, for so long as the tumor is under control and the patient tolerates the regimen or tumor regression. Seven, fourteen and fifteen day treatment schedules are preferably repeated every twenty eight days. Preferably, these treatment cycles are repeated for a total of up to about eight cycles (that is a total of about twenty four or about thirty two weeks).

In a particular embodiment, the present invention relates to a method of treating a patient suffering from cancer, in particular a solid cancerous tumor, comprising administering to the patient:

(i) a first component consisting of pharmaceutical composition containing as an active ingredient a compound of formula:

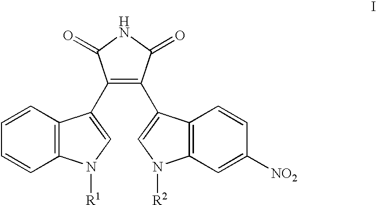

or a pharmaceutically acceptable salt or ester of said compound, wherein

R¹ is selected from the group consisting of —H, —CH₃, and —CH₂OH, and

R² is —CH₃, and

wherein the active ingredient of the first component is administered daily as an oral sustained release formulation for an administration period of up to about 14 days, in a total amount of from about 780 mg/m² to about 3000 mg/m² divided over the administration period; and

(ii) a second component consisting of a pharmaceutical composition containing as an active ingredient gemcitabine, wherein the gemcitabine is administered in a total amount of from about 1200 mg/m² to about 3600 mg/m², over about 15 days, beginning on the first day of the 21-28 day cycle;

said treatment cycle being repeated every 21-28 days for as long as the tumor remains under control and the regimen is clinically tolerated.

In another embodiment, the present invention relates to a method of treating a patient suffering from cancer, in particular a solid cancerous tumor, comprising administering to the patient:

(i) a component consisting of a pharmaceutical composition containing as an active ingredient gemcitabine, wherein the gemcitabine is administered in a total amount of from about 1200 mg/m² to about 3600 mg/m², over about 15 days, beginning on the first day of the 21-28 day cycle; and

(ii) another component consisting of pharmaceutical composition containing as an active ingredient a compound of formula:

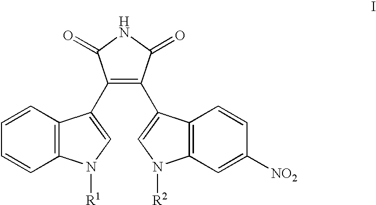

or a pharmaceutically acceptable salt or ester of said compound, wherein

R¹ is selected from the group consisting of —H, —CH₃, and —CH₂OH, and

R² is —CH₃, and

wherein the compound of formula I in the above component is administered daily as an oral sustained release formulation for an administration period of up to about 14 days, in a total amount of from about 780 mg/m² to about 3000 mg/m² divided over the administration period,

said treatment cycle being repeated every 21-28 days for as long as the tumor remains under control and the regimen is clinically tolerated.

In another embodiment, the invention relates to antitumor pharmaceutical compositions comprising at least one compound of formula I as defined above, or a pharmaceutically acceptable salt or ester of said compound, in combination with gemcitabine. According to the invention, the constituents of the combination, that is a compound of formula I and gemcitabine, may be administered simultaneously or separately space out over a period of time.

A preferred compound of formula I is:

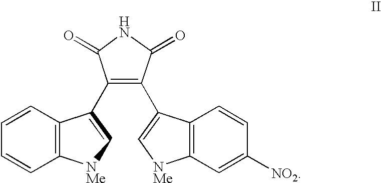

This is a known compound. See U.S. Pat. No. 5,057,614, which is incorporated herein by reference.

Other preferred compounds of formula I are

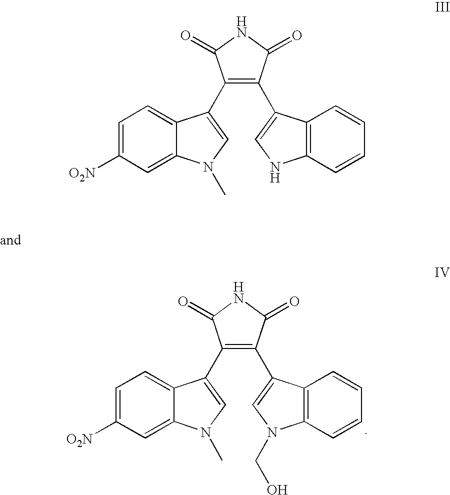

Compounds III and IV above are also known compounds. See U.S. Pat. No. 6,048,887, which is incorporated herein by reference.

The determination of tumor control (also referred to as “maintenance”) or shrinkage (also referred to as “regression”) is made by known methods. For example, by evaluation of patient symptoms, physical examination, X-ray, MRI or CAT scan or other commonly accepted evaluation modalities.

In a most preferred embodiment, from about 95 mg/m² to about 285 mg/m² of Compound II are administered twice daily (total daily dose of from about 190 mg/m² to about 570 mg/m²) for 4 consecutive days commencing on day 1 of a 21 day cycle. Also on day 1 of the cycle, preferably starting at about the same time as the first dose of Compound II, from about 700 mg/m² to about 1200 mg/m² of gemcitabine are administered as an i.v. infusion. The gemcitabine dose is repeated on day 8 of the cycle. This treatment is repeated every twenty one days, or as soon as permitted by recovery from toxicity, for so long as the tumor is under control and the patient tolerates the regimen or tumor regression. Preferably, the cycles are repeated for a total of up to 8 cycles (that is twenty four weeks).

In another most preferred embodiment, from about 135 mg/m² to about 225 mg/m² of Compound II are administered twice daily (total daily dose of from about 270 mg/m² to about 450 mg/m²) for 4 consecutive days commencing on day 1 of a 21 day cycle. Also on day 1 of the cycle, preferably starting at the about the same time as the first dose of Compound II, from about 800 mg/M² to about 1000 mg/M² of gemcitabine are administered as an i.v. infusion. The gemcitabine dose is repeated on day 8 of the cycle. This treatment is repeated every twenty one days, or as soon as permitted by recovery from toxicity, for so long as the tumor is under control and the patient tolerates the regimen or tumor regression. Preferably, the cycles are repeated for a total of up to 8 cycles (that twenty four weeks).

In another most preferred embodiment, from about 70 mg/m² to about 215 mg/m² of Compound II are administered twice daily (total daily dose of from about 200 mg/M² to about 340 mg/M²) for 7 consecutive days commencing on day 1 of a 28 day cycle. Also on day 1 of the cycle, preferably starting at the about the same time as the first dose of Compound II, from about 600 mg/m² to about 1200 mg/m² of gemcitabine are administered as an i.v. infusion. The gemcitabine dose is repeated on day 8 of the cycle. This treatment is repeated every 28 days, or as soon as permitted by recovery from toxicity, for so long as the tumor is under control and the patient tolerates the regimen or tumor regression. Preferably, the cycles are repeated for a total of up to 8 cycles (that is thirty two weeks).

In another most preferred embodiment, from about 100 mg/M² to about 170 mg/m² of Compound II are administered twice daily (total daily dose of from about 200 mg/M² to about 340 mg/m²) for 7 consecutive days commencing on day 1 of a 28 day cycle. Also on day 1 of the cycle, preferably starting at the about the same time as the first dose of Compound II, from about 800 mg/M² to about 1000 mg/m² of gemcitabine are administered as an i.v. infusion. The gemcitabine dose is repeated on day 8 of the cycle. This treatment is repeated every 28 days, or as soon as permitted by recovery from toxicity, for so long as the tumor is under control and the patient tolerates the regimen or tumor regression. Preferably, the cycles are repeated for a total of up to 8 cycles (that is thirty two weeks).

In another most preferred embodiment, from about 35 mg/m² to about 110 mg/m² of Compound II are administered twice daily (total daily dose of from about 70 mg/m² to about 220 mg/m²) for 14 consecutive days commencing on day 1 of a 28 day cycle. Also on day 1 of the cycle, preferably starting at the about the same time as the first dose of Compound II, from about 800 mg/M² to about 1000 mg/M² of gemcitabine are administered as an i.v. infusion. The gemcitabine dose is repeated on days 8 and 15 of the cycle. This treatment is repeated every 28 days, or as soon as permitted by recovery from toxicity, for so long as the tumor is under control and the patient tolerates the regimen or tumor regression. Preferably, the cycles are repeated for a total of up to 8 cycles (that is thirty two weeks).

In another most preferred embodiment, from about 50 mg/M² to about 85 mg/m² of Compound II are administered twice daily (total daily dose of from about 100 mg/m² to about 170 mg/m²) for 14 consecutive days commencing on day 1 of a 28 day cycle. Also on day 1 of the cycle, preferably starting at the about the same time as the first dose of Compound II, from about 800 mg/m² to about 1000 mg/m² of gemcitabine are administered as an i.v. infusion. The gemcitabine dose is repeated on days 8 and 15 of the cycle. This treatment is repeated every 28 days, or as soon as permitted by recovery from toxicity, for so long as the tumor is under control and the patient tolerates the regimen or tumor regression. Preferably, the cycles are repeated for a total of up to 8 cycles (that is thirty two weeks).

In accordance with the present invention, a kit useful for treating cancer is also provided. The kit comprises a first component and a second component. The first component contains one or more oral unit dosage forms, preferably capsules, of an active ingredient, each unit containing from about 50 mg to about 200 mg of the active ingredient, wherein the active ingredient is a compound of formula I. The second component contains a vial containing one or more unit dosage forms of gemcitabine as an active ingredient, each unit containing about 200 mg to about 1 g of gemcitabine.

Preferably, the first component contains a sufficient number of units so that a patient can administer up to about 600 mg per day of the active ingredient for a period of about four to 14 days and the second component contains a sufficient number of doses so that a patient can administer up to 2600 mg per day for a period of about 3 days.

In another aspect of this invention, the two components herein described above are administered in combination with radiotherapy or alternatively together with another anticancer agent.

The present invention may be exemplified by the Examples below, which illustrate the invention without limitation.

### EXAMPLES

The efficacy of the combinations of the present invention on solid tumors is demonstrated by the following experiments:

### Example 1

### In Vitro Assay

Description of Tumor Cell Lines and Cultures:

H1299 cells were obtained from the National Cancer Institute. A549 cells were purchased from ATCC. The cells were grown in a subconfluent condition to maintain a logarithmic growth phase. The cells were dislodged for passage using trypsin-EDTA (0.05% and 0.53 mM, respectively).

Tetrazolium Dye Proliferation Assay:

Cellular proliferation was evaluated by the tetrazolium dye assay. Cells from exponentially growing cultures were plated at the appropriate seeding density in the optimal medium to give logarithmic growth over the course of the assay. Plates were incubated overnight at 37° C. in a humidified CO₂ atmosphere to allow for recovery from trypsinization before beginning the assay. Compound II stock solution was prepared in DMSO (dimethyl sulfoxide) and aliquots were stored at −20° C. Purified gemcitabine was diluted in sterile saline. Each drug was diluted to eight times the final concentration in media containing 0.4% DMSO. One eighth final well volume of each dilution was added in triplicate to plates containing cells. An equal volume of 0.4% DMSO in media was added to single drug wells and twice the volume of 0.4% DMSO in media was added to a row of control wells so that the final concentration of DMSO in all wells was 0.2%. The plates were returned to 37° C. and assayed for proliferation by MTT (3-(4,5-dimethyl-thiazole-2-yl)-2,5-diphenyl-2H-tetrazolium bromide at time points that would allow for at least three population doublings in the untreated control cultures. MTT was added to each well to yield a final concentration of 1 mg/ml and the plates were returned to 37° C. for 2.5 hours. Following incubation, the MTT-containing medium was removed by aspiration and the resulting formazan metabolite was solubilized in 50 μl ethanol. Absorbances were read at a wavelength of 570 nm with a 650 nm reference. Percent inhibition was calculated using the formula:

$$Percent Inhibition = 1 - [ \frac{Mean Absorbance of Experimental Wells}{Mean Absorbance of Control Wells} ] \times 100$$

Findings:

The antiproliferative activity of Compound II in combination with gemcitabine was evaluated in vitro using a tetrazolium dye assay in two different tumor cell lines derived from non-small cell lung cancers. Table 3 below shows that in cell culture studies with A549 (non-small cell lung carcinoma) and H1299 (non-small cell lung carcinoma) tumor cells, Compound II in combination with gemcitabine produced a statistically significant greater growth inhibitory effect than that produced by either compound alone at the same concentrations. The in vitro studies demonstrate dose combinations of Compound II with gemcitabine that provide superior antiproliferative activity compared to corresponding doses of these same agents in monotherapy.

TABLE 3
Statistical comparisons for various combinations of Compound II in combination
with Gemcitabine in vitro

| | Percent | | | | |
|---|---|---|---|---|---|
| | Inhibition | Percent Inhibition | Percent Inhibition | Compound II vs | Gemcitabine vs |
| | In Compound II | In Gemcitabine | In Combination | Combination | Combination |
| Cell line | Treated Cultures | Treated Cultures | Treated Cultures | p value* | p value |
| H1299 | 38.9 ± 5.10 | 43.0 ± 0.09 | 81.6 ± 8.16 | 0.002 | 0.001 |
| | 38.9 ± 5.10 | 43.5 ± 2.30 | 68.9 ± 2.75 | <0.001 | <0.001 |
| | 40.0 ± 1.36 | 43.0 ± 0.09 | 82.2 ± 7.80 | <0.001 | <0.001 |
| A549 | 44.2 ± 1.24 | 42.0 ± 0.88 | 65.1 ± 7.94 | 0.011 | 0.007 |
| | 44.2 ± 1.24 | 40.2 ± 1.81 | 55.3 ± 1.71 | <0.001 | <0.001 |
| | 47.9 ± 1.44 | 42.0 ± 0.88 | 63.9 ± 1.47 | <0.001 | <0.001 |

*P values for various combinations of Compound II and gemcitabine were determined using the unpaired t-test (SigmaStat) to compare triplicate values of Compound II-treated cultures to triplicate values of combination-treated cultures and triplicate values of gemcitabine-treated cultures to triplicate values of combination-treated cultures.

### Example 2

### In Vivo Assay

In Vivo Implantation:

For implantation in mice, A549 cells were dislodged with trypsin-EDTA, washed with 1×D-PBS, and resuspended in serum-free media. Tumor cells were implanted subcutaneously in mice using a 27 gauge needle and 1 ml syringe. Tumors were allowed to establish until they were palpable and of an appropriate tumor volume (based on previous tumorigenicity studies) before initiation of treatment. Animals were randomly assigned to groups according to initial tumor volume, and the day of tumor cell inoculation was considered to be study day 0.

Mice:

Female BALB/c nu/nu mice were obtained from Charles River Laboratories (Wilmington, Mass.). The mice were housed in an AALAC approved facility and received standard care in accordance with institutional policy (fully compliant with both NIH and Roche Animal Care and Use Committee guidelines).

Drug Preparation and Treatment:

Compound II was microprecipitated with an ionic polymer, Eudragit L-100-55, using a solvent extraction method. Eudragit L100-55 (described in USP/NF as “Methacrylic Acid Copolymer”) is an anionic copolymer based on methacrylic acid and ethyl acrylate. The ratio of drug to polymer in the final formulation was 50% w/w Compound II and 50% w/w Eudragit L100-55. For oral administration, the formulated Compound II or pure Eudragit L100-55 (administered to vehicle control groups) was suspended in 0.2% CMC (carboxy-methyl cellulose) solution (Aqualon Hercules, Inc., Parlin, N.J.). The CMC solution was added immediately prior to dosing to animals. Gemzar® (Lilly, Indianapolis, Ind.) was applied to a 40 micron high performance Whatman C-18 column. The column was eluted at 60 ml/min for 50 minutes with water, followed by elution with 40% CH₃CN/water until UV clear. The solution was then evaporated and lyophilized. The purified gemcitabine had identical potency to Gemzar® in MTT assay using H1299 cells (IC₅₀ 3.8 nM/IC₉₀ 5.9 nM for Gemzar® versus IC₅₀ 3.8 nM/IC₉₀ 6.0 nM for purified gemcitabine).

Measurements and Statistical Analysis:

Weight loss was graphically represented as percent change in mean group body weight, using the formula:

((W−W₀)/W₀)×100,

where ‘W’ represents mean body weight of the treated group at a particular day, and ‘W₀’ represents mean body weight of the same treated group at initiation of treatment. Maximum weight loss was also represented using the above formula, and indicated the maximum percent body weight loss that was observed at any time during the entire experiment for a particular group.

Efficacy data was graphically represented as the mean tumor volume ±standard error of the mean (SEM). Tumor volumes of treated groups were presented as percentages of tumor volumes of the control groups (%T/C), using the formula:

100×((T−T₀)/(C−C₀)),

where ‘T’ represented mean tumor volume of a treated group on a specific day during the experiment, ‘T₀’ represented mean tumor volume of the same treated group on the first day of treatment; ‘C’ represented mean tumor volume of a control group on a specific day during the experiment, and C₀′ represented mean tumor volume of the same treated group on the first day of treatment.

Tumor volume (mm³) was calculated using the ellipsoid formula:

(D×(d²))/2,

where ‘D’ represents the large diameter of the tumor, and ‘d’ represents the small diameter. In some cases, tumor regression and/or percent change in tumor volume was calculated using the formula:

((T−T₀)/T₀)×100,

where ‘T’ represents mean tumor volume of the treated group at a particular day, and ‘T₀’ represents mean tumor volume of the same treated group at initiation of treatment.

Statistical analysis was determined by the rank sum test (SigmaStat, v.2.0, Jandel Scientific, San Francisco, Calif.). Differences between control and experimental groups were considered to be significant when the probability value (p) was <0.05.

Findings:

Table 4 below shows that significant efficacy (tumor growth inhibition) was produced by combining low doses of Compound II and gemcitabine which were less effective as single agents. This particular combination was well tolerated, showing little evidence of enhanced toxicity. The in vivo studies demonstrate dose combinations of Compound II with gemcitabine that provide superior therapeutic index compared to corresponding regimens using these same agents in monotherapy.

TABLE 4
Efficacy and Toxicity of Compound II in Combination with
Gemcitabine in the A549 NSCLC Xenograft Model

| | | | | | | | | |
|---|---|---|---|---|---|---|---|---|
| | | Tumor Volume | Tumor Volume | | | Change in | Maximum | Drug |
| | | (Mean ± SEM) | (Mean ± SEM) | | | Body Weight | Weight | Related |
| | | mm3 | mm3 | % T/C* | p values | (Day 47) | Loss | Deaths |
| Group | n Route | (Day 30) | (Day 47) | (Day 47) | (Day 47) | (%) | (%) | (%) |

SINGLE AGENTS

| | | | | | | | | |
|---|---|---|---|---|---|---|---|---|
| Vehicle (Eudragit/CMC) | 9 PO | 110 ± 16 | 337 ± 82 | — | — | 2 | 0 | 0 |
| CII**: 50 mg/kg | 9 PO | 104 ± 18 | 217 ± 32 | 50 | 0.470 | 5 | 0 | 0 |
| CII: 100 mg/kg | 9 PO | 148 ± 28 | 213 ± 35 | 29 | 0.121 | 6 | 0 | 0 |
| CII: 200 mg/kg | 9 PO | 99 ± 17 | 141 ± 21 | 18 | 0.008 | 4 | 0 | 0 |
| Vehicle (Saline) | 9 IP | 117 ± 23 | 273 ± 62 | — | — | 6 | 0 | 0 |
| G***: 30 mg/kg | 10 IP | 97 ± 15 | 210 ± 44 | 72 | 0.427 | 5 | 0 | 0 |
| G: 60 mg/kg | 10 IP | 111 ± 17 | 188 ± 27 | 49 | 0.206 | 6 | 0 | 0 |
| G: 120 mg/kg | 10 IP | 98 ± 20 | 193 ± 36 | 61 | 0.253 | 7 | 0 | 0 |
| SIMULTANEOUS COMBINATION | | | | | | | | |
| Vehicle (Eudragit/CMC & Saline) | 9 PO/IP | 106 ± 21 | 243 ± 42 | — | — | 2 | 0 | 0 |
| CII: 50 mg/kg & G:30 mg/kg | 0 PO/IP | 142 ± 24 | 217 ± 42 | 55 | 0.111 | 5 | 0 | 0 |
| CII: 100 mg/kg & G:30 mg/kg | 0 PO/IP | 115 ± 9 | 192 ± 28 | 57 | 0.094 | 6 | 0 | 0 |
| CII: 100 mg/kg & G:60 mg/kg | 0 PO/IP | 125 ± 26 | 190 ± 30 | 48 | 0.037 | 5 | 0 | 0 |
| CII: 200 mg/kg & G:120 mg/kg | 0 PO/IP | 119 ± 17 | 146 ± 20 | 19 | 0.002 | −8 | −13 (Day 45) | 0 |

FORWARD SEQUENCE COMBINATIONS
(Compound II followed by Gemcitabine)

| | | | | | | | | |
|---|---|---|---|---|---|---|---|---|
| Vehicle (Eudragit/CMC & Saline) | 9 PO/IP | 106 ± 21 | 243 ± 42 | — | — | 2 | 0 | 0 |
| CII: 50 mg/kg & G: 30 mg/kg | 0 PO/IP | 98 ± 12 | 182 ± 42 | 62 | 0.361 | 7 | 0 | 0 |
| CII: 100 mg/kg & G: 30 mg/kg | 0 PO/IP | 101 ± 16 | 142 ± 27 | 30 | 0.006 | 7 | −1 (Day 32) | 0 |
| CII: 100 mg/kg & G: 60 mg/kg | 0 PO/IP | 110 ± 16 | 134 ± 15 | 18 | 0.001 | 7 | 0 | 0 |
| CII: 200 mg/kg & G: 120 mg/kg | 0 PO/IP | 125 ± 22 | 175 ± 55 | 36 | 0.017 | −5 | −13 (Day 38) | 50 |

REVERSE SEQUENCE COMBINATIONS
(Gemcitabine followed by Compound II)

| | | | | | | | | |
|---|---|---|---|---|---|---|---|---|
| Vehicle (Eudragit/CMC & Saline) | 9 PO/IP | 106 ± 21 | 243 ± 42 | — | — | 2 | 0 | 0 |
| G: 30 mg/kg & CII: 50 mg/kg | 0 IP/PO | 122 ± 24 | 144 ± 28 | 16 | 0.005 | 3 | −1 (Day 32) | 0 |
| G: 30 mg/kg & CII: 100 mg/kg | 0 IP/PO | 120 ± 17 | 155 ± 26 | 25 | 0.005 | 3 | 0 | 0 |
| G: 60 mg/kg & CII: 100 mg/kg | 0 IP/PO | 102 ± 17 | 130 ± 27 | 21 | 0.010 | 1 | −3 (Day 44) | 0 |
| G: 120 mg/kg & CII: 200 mg/kg | 0 IP/PO | 97 ± 8 | 137 ± 17 | 29 | 0.014 | −4 | −18 (Day 43) | 20 |

*% T/C values were compared to the appropriate vehicle group.
**CII = Compound II
***G = Gemcitabine

The above in vitro and in vivo data identify dose combinations of Compound II and gemcitabine that are efficacious with minimal toxicity, and that are statistically superior in terms of antiproliferative activity and/or efficacy to corresponding doses of each agent used in monotherapy.

1. A method of treating a patient suffering form cancer, comprising administering to the patient a therapeutically effective amount of a combination of a first component consisting of pharmaceutical composition containing as an active ingredient a compound of formula I 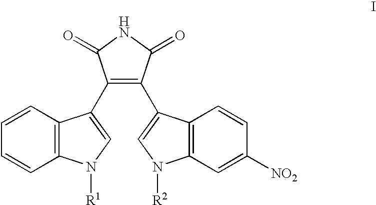
or a pharmaceutically acceptable salt or ester of said compound, wherein
R¹ is selected from the group consisting of —H, —CH₃, and —CH₂OH, and
R² is —CH₃;
and a second component consisting of gemcitabine.

2. The method of claim 1, wherein both components are administered concomitantly.

3. The method of claim 1, wherein both components are administered sequentially.

4. The method of claim 1 wherein the amount of compound of formula I in the combination is not by itself a therapeutically effective amount.

5. The method of claim 1 wherein the amount of gemcitabine I in the combination is not by itself a therapeutically effective amount.

6. The method of claim 1, wherein the pharmaceutical composition of the first component is an oral unit dosage form.

7. The method of claim 6, wherein the active ingredient of the first component is a compound of the formula: 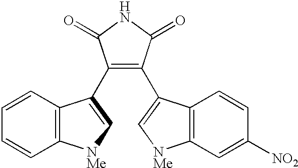
or a pharmaceutically acceptable salt or ester thereof.

8. The method of claim 6, wherein the active ingredient of the first component is a compound of the formula 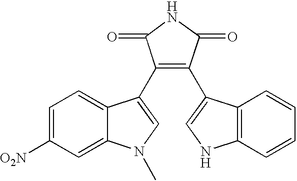
or a pharmaceutically acceptable salt or ester thereof.

9. The method of claim 6, wherein the active ingredient of the first component is a compound of the formula 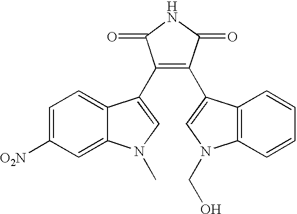
or a pharmaceutically acceptable salt or ester thereof.

10. The method of claim 1 wherein the amount of a compound of formula I is from about 1040 mg/m² to about 3000 mg/m² administered over a period of up to about 14 days.

11. The method of claim 10 wherein the amount of a compound of formula I is from about 1480 mg/m² to about 2360 mg/m².

12. The method of claim 1 wherein the amount of a compound of formula I is from about 1040 mg/m² to about 3000 mg/m² over a period of about 7 days.

13. The method of claim 12 wherein the amount of a compound of formula I is from about 1480 mg/m² to about 2360 mg/m².

14. The method of claim 1 wherein the amount of a compound of formula I is from about 1780 mg/m² to about 2250 mg/m² over a period of about 4 days.

15. The method of claim 14 wherein the amount of a compound of formula I is from about 1110 mg/m² to about 1770 mg/m² over a period of about 4 days.

16. The method of claim 1 wherein the dose intensity of the compound of formula I is from about 260 mg/m²/week to about 750 mg/m²/week.

17. The method of claim 16 wherein the dose intensity of the compound of formula I is from about 370 mg/m²/week to about 590 mg/m²/week.

18. The method of claim 1 wherein the active ingredient of the second component is gemcitabine.

19. The method of claim 18 wherein the amount of gemcitabine is from about 1200 mg/m² to about 2400 mg/m² administered over a period of up to about 8 days.

20. The method of claim 19 wherein the amount of gemcitabine is from about 1600 mg/m² to about 2000 mg/m² administered over a period of up to about 8 days.

21. The method of claim 1 wherein the amount of gemcitabine is from about 1800 mg/m² to about 3600 administered over a period of up to about 15 days.

22. The method of claim 21 wherein the amount of gemcitabine is from about 2400 mg/m² to about 300 administered over a period of up to about 15 days.

23. The method of claim 18 wherein the gemcitabine is administered on days 1 and 8 of a 21-day treatment cycle.

24. The method of claim 23 wherein the dose intensity of gemcitabine is from about 460 mg/m²/week to about 800 mg/m²/week.

25. The method of claim 24 wherein the dose intensity of gemcitabine is from about 530 mg/m²/week to about 670 mg/m²/week.

26. The method of claim 20 wherein the gemcitabine is administered on days 1 and 8 of a 21-28 day treatment cycle.

27. The method of claim 21 wherein the gemcitabine is administered on days 1, 8 and 15 of a 28-day treatment cycle.

28. A method of treating a patient suffering with cancer comprising administering to the patient:
(i) a first component consisting of pharmaceutical composition containing as an active ingredient a compound of formula: 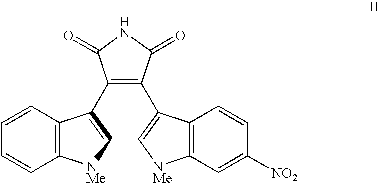
or a pharmaceutically acceptable salt or ester of said compound,
wherein the compound of formula II is administered in an amount of from about 70 mg/m² per day to about 220 mg/m² per day for up to about 14 days starting on the first day of a 28 day cycle, and
(ii) a second component consisting of an injection solution containing as an active ingredient gemcitabine which is administered in amount of from about 800 mg/m² to about 1000 mg/m² on the first, eighth and fifteenth day of a 28 day cycle, said 28 day cycle being repeated as long as the tumor remains under control.

29. A method of treating a patient suffering with cancer comprising administering to the patient:
(i) a first component consisting of pharmaceutical composition containing as an active ingredient a compound of formula: 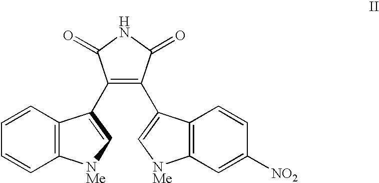
or a pharmaceutically acceptable salt or ester of said compound,
wherein the compound of formula II is administered in an amount of from about 200 mg/m² per day to about 340 mg/m² per day for up to about 7 days starting on the first day of a 28 day cycle, and
(ii) a second component consisting of an injection solution containing as an active ingredient gemcitabine which is administered in amount of from about 600 mg/m² to about 1200 mg/m² on the first and eighth day of a 28 day cycle, and said 28 day cycle being repeated as long as the tumor remains under control.

30. A method of treating a patient suffering with cancer comprising administering to the patient:
(i) a first component consisting of pharmaceutical composition containing as an active ingredient a compound of formula: 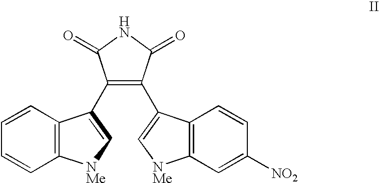
or a pharmaceutically acceptable salt or ester of said compound,
wherein the compound of formula II is administered in an amount of from about 270 mg/m² per day to about 450 mg/m² per day for up to about 4 days starting on the first day of a 21 day cycle, and
(ii) a second component consisting of an injection solution containing as an active ingredient gemcitabine which is administered in amount of from about 800 mg/m² to about 1000 mg/m² on the first and eighth day of a 21 day cycle, and said 21 day cycle being repeated as long as the tumor remains under control.

31. A method of treating a patient suffering with cancer comprising administering to the patient:
(i) a first component consisting of pharmaceutical composition containing as an active ingredient a compound of formula: 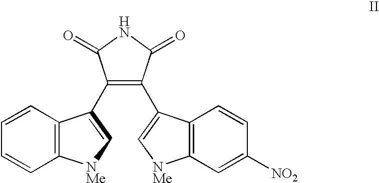
or a pharmaceutically acceptable salt or ester of said compound,
wherein the compound of formula II is administered in an amount of from about 190 mg/m² per day to about 570 mg/m² per day for up to about 4 days starting on the first day of a 21 day cycle, and
(ii) a second component consisting of an injection solution containing as an active ingredient gemcitabine which is administered in amount of from about 700 mg/m² to about 1200 mg/m² on the first and eighth day of a 21 day cycle, said 21 day cycle being repeated as long as the tumor remains under control.

32. A kit comprising:
(a) a first component containing one or more oral unit dosage forms of an active ingredient, each unit containing about 50 mg to about 200 mg of the active ingredient, wherein the active ingredient is a compound selected from formula I 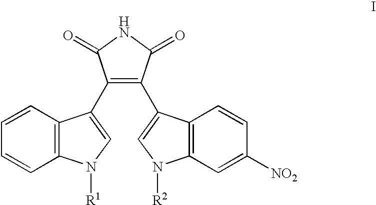
or a pharmaceutically acceptable salt or ester said compound, wherein
R¹ is selected from the group consisting of —H, —CH₃, and —CH₂OH, and
R² is —CH₃; and
(b) a second component containing a vial or series of vials, each vial containing a single injectable solution dose or multiple injectable solution doses, each dose containing as an active ingredient about 200 mg to about 1 g of gemcitabine.

33. The kit of claim 30, wherein the first component contains a sufficient number of units so that a patient can administer about 600 mg per day of the compound of formula I or a pharmaceutically acceptable salt or ester of said compound for a period of about 4 to about 14 days and the second component contains a sufficient number of doses so that a patient can administer about 2600 mg per day of gemcitabine for a period of about three days.

34. The kit of claim 33 wherein the active ingredient of the first component is

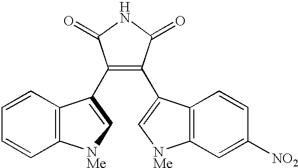

35. The kit of claim 33, wherein the active ingredient of the first component is

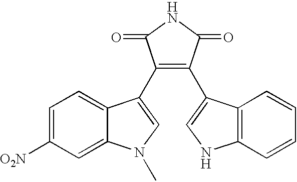

36. The kit of claim 33, wherein the active ingredient of the first component is a compound of the formula

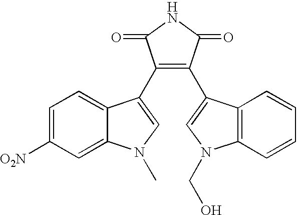

37. The method of claim 1 comprising additionally subjecting the patient to radiotherapy.
